(19) United States
(12) Patent Application Publication
(10) Pub. No.: US 2009/0136953 A1
(43) Pub. Date: May 28, 2009

(54) MOLECULAR DIAGNOSTIC METHOD FOR DETERMINING THE RESISTANCE OF A MICROORGANISM TO AN ANTIBIOTIC

(75) Inventors: Larry Gold, Boulder, CO (US); Scott D. Conlin, Longmont, CO (US)

Correspondence Address: PATTON BOGGS LLP, 1801 CALFORNIA STREET, SUITE 4900, DENVER, CO, 80202, US

(73) Assignee: MicroPhageTM Incorporated, Longmont, CO (US)

(21) Appl. No.: 12/269,753

(22) Filed: Nov. 12, 2008

Related U.S. Application Data
(60) Provisional application No. 60/987,209, filed on Nov. 12, 2007.

(51) Int. Cl. C12Q 1/68; C12Q 1/18

(52) U.S. Cl. 435/6; 435/32

(57) ABSTRACT

A sample (103) containing a microorganism is divided into a first sample portion (107) and a second sample portion (113), a growth medium i(109, 114) is added to both sample portions and an antibiotic (110) is combined with the second sample portion. The two portions are incubated and then molecular analysis, such as a polymerase chain reaction, is performed on each of the first and second sample portions to determine the resistance or susceptibility of the microorganism to the antibiotic. The concentration of the microorganism prior to the incubation can be below the detection limit or above the detection limit. If of both the portions test positive for microbe growth, the microbe is resistant. If only the portion with the antibiotic tests negative for growth, then the microbe is susceptible.

### CROSS REFERENCE TO RELATED APPLICATION

This application claims benefit to U.S. provisional application No. 60/987,209 filed Nov. 12, 2007

### BACKGROUND OF THE INVENTION

1. Field of the Invention

The invention relates generally to the field of molecular diagnostics and more particularly to the use of molecular diagnostics for identification of the molecular structure specific to particular microorganisms.

2. Statement of the Problem

Organisms resistant to antibiotics have become a significant problem over the last quarter-century. For example, the rate of methicillin-resistant Staphylococcus aureus (MRSA) has increased significantly over the last decade. Studies of patients with MRSA bacteremia have reported higher mortality rates, increased morbidity, longer hospital length of stay (LOS), and higher costs compared with patients with methicillin-susceptible S. aureus (MSSA) bacteremia. Thus, there is currently a great amount of research on methods for determining the antibiotic resistance or antibiotic susceptibility of microorganisms. Molecular diagnostic techniques that categorize microorganisms by identifying particular molecular structures, such as polymerase chain reaction (PCR) and molecular probes, have been proposed as solutions to this problem. In molecular diagnostics, the molecular structure, particularly the genetic structure, of the microorganism, is determined; and particular known genetic structures can be identified. Particular genetic markers have been shown to be indicative of resistance to particular antibiotics. If one of these markers is present, then it can be reliably assumed that the microorganism is resistant to the particular antibiotics. However, the converse is not necessarily true. That is, the absence of a resistance marker is not indicative of the susceptibility of the microorganism. Moreover, molecular diagnostics generally cannot determine if the present bacteria is viable and persisting, or has, for example, been eliminated by antibiotic or chemical means. The reason for this is that the RNA and DNA of the bacteria still exists and therefore can be copied using a typical molecular amplification method, such as PCR, used in molecular diagnostics. For these reasons, a commercially viable molecular diagnostic method of determining antibiotic susceptibility is not presently available.

Thus, it would be highly desirable if a molecular diagnostic/identification method and apparatus could be found that was more definitely determinative of antibiotic resistance and/or susceptibility. If this method and apparatus also could differentiate between live versus dead microorganisms, it would be a significant advance in the art.

### SUMMARY OF THE INVENTION

The invention solves the above problems, as well as other problems of the prior art, by combining differential culture with one or more molecular identification methods. Differential culture is any method that utilizes a difference in microorganism growth. The differential growth is unambiguously determinative of both the resistance or susceptibility of the microorganism to an antibiotic and the viability, i.e., the living or dead state, of the microorganism, while the molecular identification is determinative of the presence of a particular microorganism, preferably a bacterium. By combining the two, the antibiotic resistance or susceptibility of a particular microorganism can be reliably determined, both qualitatively and quantitatively.

The invention provides a method of determining the resistance or susceptibility of an microorganism to an antibiotic, the method comprising: (a) creating a first sample portion and a second sample portion, the first and second sample portions being essentially identical with respect to the quantity of the microorganism in the first and second samples and all other materials and conditions related to growth of the microorganism; (b) combining the antibiotic with the second sample portion; (c) providing conditions for the first sample portion and the second sample portion to support growth of the microorganism; and (d) performing molecular analysis on the first and second sample portions to determine the amount of a particular molecular structure associated with the microorganism present in each of the first and second sample portions and using the results of the molecular analysis to determine differential growth and the resistance or susceptibility of the microorganism to the antibiotic. Preferably, the concentration of the microorganism prior to the providing conditions is below the detection limit of the molecular analysis. Preferably, the performing is done after a predetermined time period. Preferably, the molecular analysis comprises amplification of a molecular structure of the microorganism. Preferably, the molecular analysis comprises a polymerase chain reaction (PCR). Preferably, the molecular analysis comprises comparison of the results of the molecular analysis for the first portion with the results of the molecular analysis for the second portion. Preferably, the creating comprises dividing an initial sample into a plurality of sample portions.

The inventive method not only can reliably and unambiguously determine the antibiotic resistance or susceptibility of particular organisms, but it is also fast and uses relatively economical techniques easily transferable to conventional laboratories. Numerous other features, objects, and advantages of the invention will become apparent from the following description when read in conjunction with the accompanying drawings.

### BRIEF DESCRIPTION OF THE DRAWINGS

FIG. 1 illustrates the splitting of a sample into a sample portion having no antibiotic and a sample portion including an antibiotic in the preferred embodiment of the antibiotic susceptibility test according to the invention;

FIG. 2 illustrates the incubation process according to the preferred embodiment of the invention;

FIG. 3 illustrates the results of several possible exemplary molecular assays on the sample portion of FIG. 1 that does not include an antibiotic in the preferred embodiment of the invention in which the starting level of the bacteria is below the detection threshold;

FIG. 4 illustrates the results of several possible exemplary molecular assays on the sample portion of FIG. 1 that includes an antibiotic in the preferred embodiment of the invention in which the starting level of the bacteria is below the detection threshold;

FIG. 5 illustrates the results of several possible exemplary assays on the two sample portions of FIG. 1 in the case where the starting level of bacteria is above the detection threshold and the bacteria is resistant to the antibiotic; and

FIG. 6 illustrates the results of several possible exemplary assays on the two sample portions of FIG. 1 in the case where the starting level of bacteria is above the detection threshold and the bacteria is resistant to the antibiotic.

### DETAILED DESCRIPTION OF THE PREFERRED EMBODIMENT

The invention comprises the combination of a differential growth process with a molecular detection process to determine the antibiotic resistance of a microorganism, preferably a bacterium. In the differential growth process, a plurality of samples containing the target microorganism are created. Preferably, the samples are essentially identical with respect to the quantity of said microorganism in said first and second samples and all other materials and conditions related to growth of said microorganism; this can be most advantageously done by dividing an initial sample into a plurality of equal sample portions. In this preferred embodiment, a first and a second sample portion are created. Conditions are provided that promote the growth of the target microorganism. These conditions include a growth medium and maintaining both the sample portions at a suitable temperature for growth. The microorganism may be combined with the growth medium either before or after the sample is divided. The antibiotic, the resistance of which is to be determined, is combined with one of the sample portions. Both sample portions are incubated under conditions sufficient to allow, and preferably promote, growth of the microorganism. Molecular analysis is then performed on both samples to determine the antibiotic resistance. If the molecular analysis determines that there was growth in both samples, that is, the test is positive for growth in both samples, the microorganism is resistant. If there is growth only in the sample not containing the antibiotic, the microorganism is susceptible. If both samples are negative for growth, the incubation period is extended until one sample shows growth or it is determined that the microorganism is not viable.

In one embodiment of the invention, the initial concentration of the microorganism is below the detection limit of the molecular identification process. This embodiment shall be referred to herein as the “threshold technique”. In this embodiment, if any of the microorganism is detected after the incubation period, the test is positive. In another embodiment, the initial concentration of the target microorganism is above the detection limit. In this embodiment, the level of the signal in the results for the two sample portions is compared to determine resistance or susceptibility.

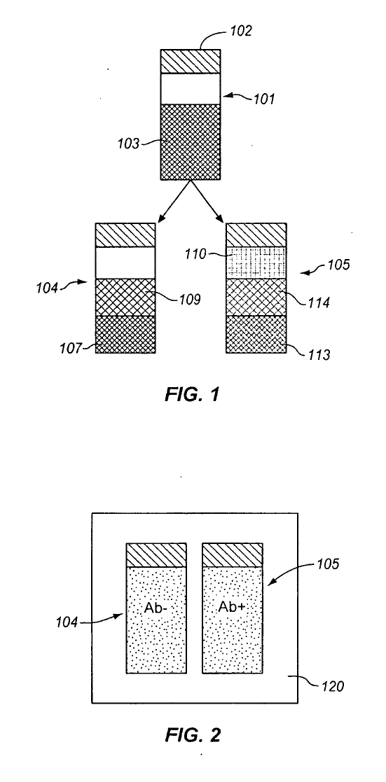

FIGS. 1 through 4 illustrate an exemplary embodiment of the invention using a threshold measurement technique. As shown in FIG. 1, the sample 101 is split into first and second reaction containers 104 and 105, respectively, each of which contains the suspect organism at a concentration that is below the concentration level at which the molecular technology can detect the microorganism. If the sample is not initially below the detection limit, it may be diluted suitably so that the concentration of the microorganism is below the detection limit. Conditions are then provided for the two sample portions to support growth of the microorganism. The conditions preferably include a growth medium 109, 114 and an appropriate temperature to support growth. As illustrated in FIG. 2, one container 104, contains a first sample portion 107 and media 109 to sustain growth. The other container 105 includes a second sample portion, 113, a media 114 to sustain growth and an antibiotic 110 for which it is desired to determine resistance/susceptibility to the antibiotic. Then, as illustrated in FIG. 2, the medium and the sample in vial 104 are thoroughly mixed to provide the sample portion Ab−, and the medium, sample and antibiotic in vial 105 are thoroughly mixed to provide the sample portion Ab+. Both sample portions are incubated in incubator 120.

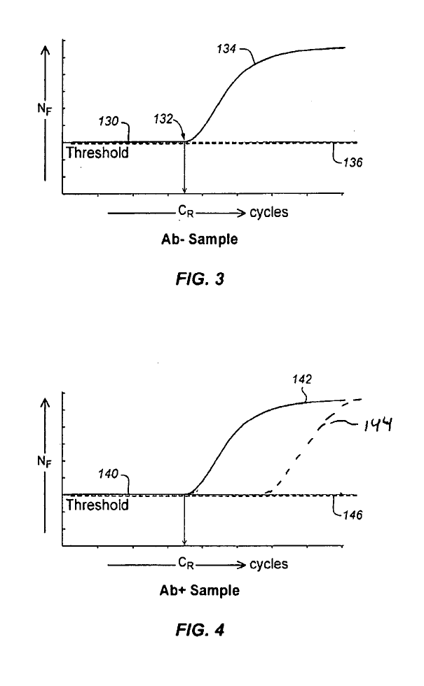

After a predetermined time period, the desired molecular analysis, such as PCR, is performed on sample portions. The time period preferably is selected so that the microorganism concentration will be well above the detection limit after the time period. Since generally, if an antibiotic resistance test is being done, it is known that a microorganism is present and at least a general idea of the concentration is known, this time period can usually be selected with a high degree of certainty. However, it is still possible to employ the invention in cases where the concentration, or even the presence, of a microorganism is not known with accuracy. Typical results of the test for an embodiment in which PCR is the selected molecular analysis, are shown in FIG. 3 for the Ab− sample and in FIG. 4 for the Ab+ sample. The curves of FIGS. 3 and 4 show the cycle number, C_{R}, along the abscissa, and the number of fluorescence units, N_{F}, along the ordinate. At first, along the portion 130 of the curve, the curve remains at threshold, as there is no detectable number of fluorescence units. Then, at the point 132, if the microorganism is present, the fluorescence begins to be detected and rises along the curve 134 as the PCR technique amplifies the microorganism nucleic acid. If the microorganism is not present, then the curve stays at threshold along line 136. Turning to FIG. 4, for the Ab+ sample portion, the number of fluorescence units at first stays at threshold along line 140. Three different exemplary curves are show at 142, 144, and 146. If the microorganism is resistant to the antibiotic, the curve 142 will be essentially the same as curve 134. If the microorganism is susceptible to the antibiotic, the curve will be shifted, as shown at 144. The displacement of the curve, provides the degree of resistance and the difference between the curves 142 and 144 or 142 and 146 gives the degree of susceptibility. This response reflects the physiology of resistance: despite being genetically identical, a group of organisms within a culture displays a certain degree of physiological heterogeneity. As a consequence, some organisms, though genetically susceptible, will display a limited degree of resistance, leading to limited growth of some cells. This growth, in turn is reflected in the output of the molecular test. If the microorganism is highly susceptible, or if the microorganism is not present, the curve may remain at threshold, as shown at 146.

After the PCR is run, the curves of FIGS. 3 and 4 are interpreted. If both sample portions are positive, as shown by curves 134 and 142, the organism is deemed antibiotic resistant. If the Ab− sample portion gives a positive result as at 134, but the Ab+ sample portion gives a significantly lower result, as at 144 or 146, the organism is deemed susceptible. If the test is negative, that is, if for both sample portions, the concentration of the microbe remains below the detection limit, the vials are incubated for an additional time. This may be repeated until the microorganism is detected in one of the vials. If after a period of time in which a living microbe would have been detected with certainty the concentration is still below the detection limit, then the test is defective, either because there was some defect or error in the process or the organism is not present or alive. Detection of resistance markers in addition to markers which identify specific microorganisms can be used to aid in confirming resistant organisms.

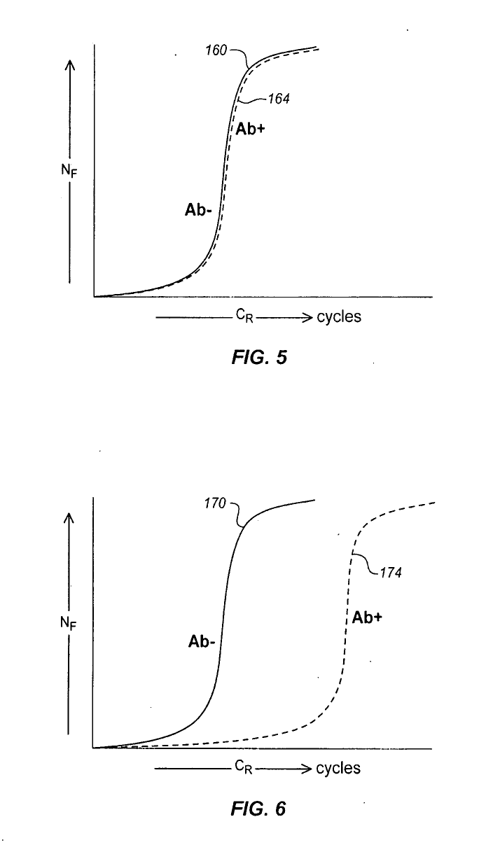

FIGS. 5 and 6 illustrate an embodiment of the invention in which the starting sample of the microorganism is above the detection limit. Again, as illustrated in FIGS. 1 and 2, the sample is split into two reaction containers, such as PCR vials, with Ab+ containing the sample, growth media and an antibiotic and Ab− containing the sample and growth media, and appropriate temperatures and possibly other additional conditions are provided to promote the growth of the microbe. The two samples are allowed to incubate for a given period of time. Then, a molecular analysis, such as PCR, is used to detect/identify the microbe, and curves for the Ab− and Ab+ sample portions are generated. Exemplary curves of fluorescence number, N_{F}, versus PCR cycle are shown in FIGS. 5 and 6. In FIG. 5, curve 160 is for the Ab− sample portion and curve 164 is for the Ab+ sample portion. The curves 160 and 164 for the Ab− and Ab+ portions, respectively, are essentially identical, which indicates that the antibiotic had no effect, and the microorganism is resistant. In FIG. 6, the Ab+ curve 174 is shifted to the right from the Ab− curve 170. The shift to the right occurs because the fluorescence number stays low for many more PCR cycles, which indicates that the number of microbes that the process started with was significantly lower. This means that the microbe did not grown as well, or did not grow at all, in the presence of the antibiotic, and the microorganism is susceptible. The Ab− curve provides a base line to determine the susceptibility or resistance, and the difference between the Ab− and Ab+ curves can be analyzed to determine the degree of resistance or susceptibility to the antibiotic in the Ab+ sample portion. This works particularly well for the RT-PCR and qPCR molecular diagnostic methods, where differentiation between curves can be made. In the curves of FIGS. 5 and 6, a shift to the right of the Ab+ curve indicates susceptibility. If the Ab− curve and the Ab+ curve are essentially the same or differ only a little, antibiotic resistance is indicated. The amount of difference between the curves can be analyzed to quantitatively determine the resistance and/or the effectiveness of the antibiotic against the particular microorganism.

To make the analysis more rapid, “standard” curves can be run with a variety of different microorganisms and antibiotics showing a variety of different resistances. Test curves then can be compared to the “standard” curves to rapidly determine the resistance/susceptibility of the target microorganism to the target antibiotic.

It is possible that, if the test is identically, or nearly identically positive for both sample portions, that the molecular analysis is detecting dead microorganisms rather than a resistant microorganism. However, in the case of a resistant microorganism, to determine an antibiotic that is effective, tests with additional antibiotics will always be run, one of which will result in at least some differential growth is the bacteria is viable.

While the invention has been disclosed in terms of the combination of a PCR process with a differential growth process, other molecular methods, such as molecular probes, mass spectrometry, and other methods of identifying molecular structure may be used.

A feature of the invention is that it provides either a definite indication of the antibiotic resistance or susceptibility of microorganism or an indication that the test was defective in some way.

Another feature of the invention is that the antibiotic resistance determination process distinguishes between live and dead bacteria. This is essential for antibiotic resistance tests or antibiotic susceptibility tests. It is also essential for food applications, where the food has been irradiated, or any other application where dead bacteria may be present. Thus, the invention provides significant advantages over other molecular analysis tests in which it is impossible or difficult to distinguish between live and dead bacteria.

There has been described an antibiotic resistance determination method which is reliable, sensitive, simple, fast, and/or economical, and having numerous novel features. It should be understood that the particular embodiments shown in the drawings and described within this specification are for purposes of example and should not be construed to limit the invention, which will be described in the claims below. For example, while the disclosure has been discussed in terms of antibiotic resistance and the PCR assay, it is evident that the methods can equally be used to determine antibiotic susceptibility and many other molecular analysis methods may be used. Further, it is evident that those skilled in the art may now make numerous uses and modifications of the specific embodiment described, without departing from the inventive concepts. Equivalent structures and processes may be substituted for the various structures and processes described; the subprocesses of the inventive method may, in some instances, be performed in a different order; or a variety of different materials and elements may be used. Consequently, the invention is to be construed as embracing each and every novel feature and novel combination of features present in and/or possessed by the microorganism detection apparatus and methods described.

We claim:

1. A method of determining the resistance or susceptibility of an microorganism to an antibiotic, said method characterized by:
(a) creating a first sample portion and a second sample portion, said first and second sample portions being essentially identical with respect to the quantity of said microorganism in said first and second samples and all other materials and conditions related to growth of said microorganism;
(b) combining said antibiotic with said second sample portion;
(c) providing conditions for said first sample portion and said second sample portion to support growth of said microorganism; and
(d) performing molecular analysis on said first and second sample portions to determine the amount of a particular molecular structure associated with said microorganism present in each of said first and second sample portions and using the results of said molecular analysis to determine differential growth and the resistance or susceptibility of said microorganism to said antibiotic.

2. A method as in claim 1 further characterized in that the concentration of said microorganism prior to said providing conditions is below the detection limit of said molecular analysis.

3. A method as in claim 2 further characterized in that said performing is done after a predetermined time period.

4. A method as in claim 1 wherein the concentration of said microorganism is initially above the detection limit of said molecular analysis and further characterized by diluting said sample so that the concentration of said microorganism is below the detection limit.

5. A method as in claim 1 further characterized in that said molecular analysis comprises amplification of a molecular structure of said microorganism.

6. A method as in claim 4 further characterized in that said molecular analysis comprises a polymerase chain reaction (PCR).

7. A method as in claim 6 wherein said PCR is RT-PCR or qPCR.

8. A method as in claim 1 further characterized in that the concentration of said microorganism prior to said providing is above the detection limit of said molecular analysis.

9. A method as in claim 6 further characterized in that said molecular analysis comprises comparison of the results of said molecular analysis for said first portion with the results of said molecular analysis for said second portion.

10. A method as in claim 1 further characterized in that said creating comprises dividing an initial sample into a plurality of sample portions.
